(19) United States
(12) Patent Application Publication
(10) Pub. No.: US 2025/0114285 A1
(43) Pub. Date: Apr. 10, 2025

(54) ARRAY OF HAIR DYE PRODUCTS

(71) Applicant: The Procter & Gamble Company, Cincinnati, OH (US)

(72) Inventors: Shufen Fan, Singapore (SG); Mei Yi Tee, Singapore (SG); Pei Qi Lim, Singapore (SG); Xin Ya Tan, Singapore (SG)

(21) Appl. No.: 18/906,341

(22) Filed: Oct. 4, 2024

Related U.S. Application Data
(60) Provisional application No. 63/588,401, filed on Oct. 6, 2023.

(51) Int. Cl. A61K 8/37; A61K 8/49; A61Q 5/10

(52) U.S. Cl. CPC A61K 8/37; A61K 8/4913; A61K 8/4986; A61Q 5/10; A61K 2800/438; A61K 2800/87; A61K 2800/884

(57) ABSTRACT

An array of hair dye products formulated to provide the same hair color to users with different hair colors or a different variety of colors of users with the same hair color. The array includes at least two hair dye products. The hair dye products each include a hair dye composition contained in a package. A first hair dye product in the array includes a photochromic dye that provides a color change to a first hair color, and a second hair dye product in the array includes the same photochromic dye that provides the same color change to a second, different color of hair. The array may also include two or more hair dye products that contain different ratios of photochromic dye(s) to provide users with different hair colors the same or different tunable hair colors.

### FIELD

The present disclosure generally relates to an array of hair dye compositions that enable users with different hair colors to tunably change the color of their hair by applying a suitable energy source to the hair dye composition. More specifically, the present disclosure relates to an array of hair dye products, wherein each hair dye product comprises a photochromic dye and is specifically tailored to provide multiple hair color changes to a particular color of hair upon the application of electromagnetic radiation.

### BACKGROUND

Altering the color of human hair has long been used to cosmetically adapt a person's appearance to the fashion and style trends of the day. Conventional hair coloring techniques typically involve the application of a temporary, semi-permanent, demi-permanent or permanent chemical dye (oxidative or non-oxidative) to the hair to achieve a color change. Conventional hair dyes can provide a wide variety of hair color changes, hair lightening effects and hair darkening effects. However, conventional hair dyes are limited to providing a single hair color change. Additional hair color changes require application of a different hair dye composition. If a user desires a return to their natural hair color, then they must wait for the dye to be washed out or, in the case of permanent dyes, for the hair to grow out and the dyed portion cut off. Thus, the use of conventional hair dyes can be inconvenient and time consuming.

To address some of the drawbacks of conventional hair dyes, photochromic dyes have been examined for color change options and flexibility. For example, FR 1,604,929 describes a hair composition that contains compounds such as nitrobenzylpyridines, thiosemicarbazones or spiropyran derivatives. When these compounds are exposed to electromagnetic radiation (“EMR”) of a particular wavelength (e.g., ultraviolet radiation with a wavelength of 100-400 nm), the dye compound undergoes a conformational change that alters the color of the dye. When the EMR is removed, the molecule reverts to its previous conformation and color. Compositions that contain thes photochromic dyes disclosed in FR 1,604,929 may provide additional flexibility and convenience for changing hair or skin color, but it would be desirable to provide a hair coloring composition that does not depend on the presence or absence of a particular wavelength of EMR to maintain the desired color change.

In another example, U.S. Pat. No. 6,123,952 discloses a cosmetic composition that contains a thermally-irreversible, photochromic coloring agent, preferably selected from diarylethene compounds and fulgide compounds. According to U.S. Pat. No. 6,123,952, after the composition is applied to a bodily surface, it is exposed to EMR of a particular wavelength (e.g., ultraviolet radiation) that causes the photochromic dye to change color, thereby imparting a color change to the bodily surface. However, when the UVR is removed the photochromic dye retains its color. When the dye is exposed to EMR having a particular wavelength that is different from the previous EMR, the dye returns to its original color. While the hair dye compositions of U.S. Pat. No. 6,123,952 may provide additional flexibility and convenience for coloring hair or skin, these compositions are still limited to a single hair color change (i.e., coloring and de-coloring).

Additionally, with conventional hair dye products, difficulties have been encountered in providing consistent color effects to hair of different colors (e.g., blond, brown and black hair). To overcome this issue, medium and darker colored hair are commonly pre-treated to lighten the color of the hair (e.g., bleaching) and then applying the desired finish color. Thus, a hair dye product that provides a consistent finish color to different colors of hair without the need for pre-treatment is needed.

US2021/0308020 describes using a thermochromic encapsulated liquid crystal to provide a tunable hair color change. In particular, the publication describes how the encapsulated liquid crystal can be tuned to provide a static color effect by using a broader bandwidth such that upon reaching the starting point a color is observed and the clearing point is above the temperature that the composition is likely to encounter. However, the drawback with thermochromic dyes is the dependence on a particular temperature or temperature range to provide a color change or multiple color changes. Thus, the options for a user to tune their hair color with a thermochromic dye may be limited relative to a photochromic dye.

Accordingly, it would be desirable to provide a lineup of tunable hair dye products that provide a consistent hair color change to users with different hair colors without the need to pretreat darker colored hair. It would also be desirable for the product lineup to enable users with different hair colors to conveniently change their hair color multiple times with a single hair dye composition.

### SUMMARY

Disclosed herein is an array of tunable hair dye products for providing the same hair color to users with different hair colors, comprising: a first hair dye product comprising a package and a dye composition contained in the package, wherein the hair dye composition comprises a p-type photochromic dye and a carrier; and a second hair dye product comprising a package and a hair dye composition contained in the package, wherein the hair dye composition comprises a p-type photochromic dye and a carrier, wherein the first and second hair dye compositions, when activated, yield different hair color changes to hair of the same color and the same color change to hair of different colors, according to the Color Measurement method.

Also disclosed is an array of tunable hair dye products formulated to provide the same tunable hair color to users who have different hair colors, comprising: a first tunable hair dye product comprising a first hair dye composition contained in a first package, wherein the first hair dye composition comprises first and second p-type photochromic dyes present at a weight ratio of first photochromic dye to second photochromic dye of 1:10 to 10:1; a second tunable hair dye product comprising a second hair dye composition contained in a second package, wherein the second hair dye composition comprises the first and second photochromic dyes present at a weight ratio that is different from the weight ratio of the first hair dye composition; wherein the first package and the second package are spatially separated from each other in a retail environment such that the first package and the second package are visible to a consumer during the consumer's purchasing decision process.

### BRIEF DESCRIPTION OF THE DRAWINGS

FIGS. 1A, 1B, and 1C are examples of photochromic dyes.

FIGS. 2A, 2B, 2C, 2D, 2E, 2F, and 2G are examples of photochromic dyes.

FIG. 3 is an example of package for a hair dye composition containing a photochromic dye.

FIG. 4 is an example of package for a hair dye composition containing a photochromic dye.

FIG. 5 illustrates activation EMR curves for p-type DAE dyes.

FIG. 6 illustrates deactivation EMR curves for p-type DAE dyes.

FIG. 7 illustrates activation times for p-type DAE dyes.

FIGS. 8A, 8B and 8C illustrate deactivation times for p-type DAE dyes.

### DETAILED DESCRIPTION

Arrays of hair dye products are known. However, conventional lineups of hair dye products may not provide consistent hair color change to users with different hair colors, especially for users with darker hair. Additionally, conventional lineups of hair dye products typically only provide one color change. While some thermochromic and photochromic dyes may enable multiple hair color changes, they may not provide the desired amount of color tuning. To overcome the drawbacks of conventional hair dye products, the array of hair dye products disclosed herein utilizes specific photochromic dye formulations to provide tunable hair colors and consistent hair color change for users with different hair colors.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges not explicitly delineated within these ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

“About” modifies a particular value by referring to a range of plus or minus 20% or less of the stated value (e.g., plus or minus 15% or less, 10% or less, or even 5% or less).

“Activate” and variations thereof, when referring to a photochromic dye or a composition comprising a photochromic dye, means causing the photochromic dye and/or composition to change color by exposing it to electromagnetic radiation (“EMR”) of a particular wavelength or changing the temperature of the dye.

“Array of hair dye products” means a group of two or more hair dye products that have at least one physical or chemical feature that distinguishes one product in the group from the other product(s) in the group, and whose packages display a common brand name and/or trademark.

“Color” refers to visibly perceived light of a particular wavelength, generally between 400 nm and 700 nm. Color can be characterized in terms of Hue, Saturation, and Lightness, and can be measured using conventional methods, for example, with a spectrophotometer or colorimeter.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the U.S. Food and Drug Administration.

“Deactivate” and variations thereof, when referring to a photochromic dye or a composition comprising a photochromic dye, means causing the photochromic dye and/or composition to revert back to substantially the same color it was prior to activation by exposing the dye to EMR of a particular wavelength, removing activating EMR or changing the temperature of the dye.

“Different colors” and variations thereof mean colors that have a different Hue, Saturation and/or Lightness value relative to one another. In some aspects, different colors can be characterized by having a ΔE of greater than 1, relative to one another, according to the Color Measurement method.

“Fluence” means the time-integrated flux of EMR (i.e., activating EMR and deactivating EMR) on an irradiated surface. Fluence can be readily determined by those skilled in the art.

“Suitable for application to human hair” means that the composition or components thereof, are acceptable for use in contact with human hair and the scalp and skin without undue toxicity, incompatibility, instability, and the like.

“Substantially free of” means a composition or ingredient contains less than 3% (e.g., less than 2%, 1% or even less than 0.5%) of a subject material, by weight of the composition or ingredient. “Free of” means 0% of a subject material is present in the composition or ingredient.

“Tunable” means that the hair color change provided by the photochromic hair dye compositions herein can be adjusted to a desired color. The hair color change may be tuned, for example, by changing: the activating/deactivating time, the strength of the EMR source, the concentration or ratio of photochromic dyes and/or the activating/deactivating technique (e.g., how close the EMR source is to the hair or selectively exposing target portions of hair to the EMR source).

“Visible color change” and variations thereof mean a color change that results in a ΔE of more than 1 (e.g., 2, 3, 5, 10, 20 or more). ΔE can be determined according to the Color Measurement method described in more detail below.

### Hair Dye Composition

The cosmetic hair dye compositions herein are suitable for application to a keratinous surface such as hair or skin and provide a convenient way for a user to tune the color of their hair between at least 3 visibly different colors, which are the original hair color, a first new color, and a second new color. The tunable hair dye compositions herein include at least two different photochromic dyes, a cosmetically acceptable solvent and a carrier, as well as other optional ingredients commonly found in hair care compositions such as hair dyes, shampoos, conditioners, hair styling products and the like. The photochromic dyes may each be present in the hair dye composition at 0.01% to 25% (e.g., 0.05% to 20%, 0.1% to 10%, 0.5% to 5%, or even 1% to 3%). The composition also includes a suitable solvent to ensure the dyes are suitably dispersed in the carrier. The solvent may be present at 1% to 99.99% (e.g., 2% to 90%, 3% to 80%, 4% to 70%, or even 5% to 50%). The pH of the hair dye compositions herein may be between 2 and 10 (e.g., 3, 4, 5, 6, 7, 8 or 9). The hair dye compositions herein may be provided in various product forms such as solutions, suspensions, shampoos, conditioners, lotions, creams, gels, sticks, sprays, aerosols, ointments, solid bars, pastes, foams, mousses, hydrogels, and the like. The composition form may follow from the particular dermatologically acceptable carrier chosen.

In some embodiments, the photochromic dyes are included in the hair dye composition at a ratio of first dye to second dye of 1:50 to 50:1 (e.g., 1:30, 1:20, 1:10, 1:5, 1:3, 1:2, 1:1, 2:1, 3:1, 5:1, 10:1, 20:1, or 30:1), depending on the desired hair color change. Of course, one or more additional photochromic dyes may also be included at a suitable ratio to the first and/or second dye. It is to be appreciated that the ratio of the photochromic dyes in the composition can be important for providing the desired hair color change to users who have different hair colors. For example, to provide the same or similar color of hair to a user with blond hair and a user with black hair, it may be necessary for the ratio of photochromic dyes in the hair dye composition to be different.

Hair color can be classified on a scale of 1 to 10 based on well-known hair color charts such as the one below.

| 1 | 2 | 3 | 4 | 5 | 6 | 7 | 8 | 9 | 10 |
|---|---|---|---|---|---|---|---|---|---|
| Lightest | Medium | Dark | Lightest | Medium- | Medium | Dark | Darkest | Black | Darkest |
| Blonde | Blonde | Blonde | Brown | light | Brown | Brown | Brown | | Black |
| | | | | Brown | | | | | |

The hair color values shown in the chart can readily be correlated to red, green, and blue color values based on the RGB color models, for example, as described in US 2021/0308020. Additionally or alternatively, the hair color values can be correlated to other known color values/models such as: cyan, magenta, yellow, black (CMYK) color values; L*a*b* color values, as defined by the International Commission on Illumination (CIE Lab); or Hue, Saturation, and Lightness (HSL) color values. By tailoring the type and amount of dye in the composition based on the color interactions of known color models, it is possible to provide a user with a more predictable hair color and a wider range of tunable color options.

Providing temporary color effects to medium or darker colored hair is known to be problematic. Conventional hair coloring systems may resort to pre-treating darker colored hair to lighten the color of the hair, of the hair, sometimes referred to as bleaching and then applying a desired final color. However, bleaching is known to be harmful to hair. By tailoring the combination of photochromic dyes to the user's hair color, the hair dye compositions described herein can provide a desired visible hair color change without the need to lighten or otherwise pre-treat the hair.

In some aspects, one or more of the hair dye products in the array may include a non-oxidizing pre-treatment agent (“primer”). For example, hair dye product(s) in the array that are intended to treat dark brown and/or black hair may include a primer that acts as a color correction layer on darker hair. In this example, the primer can enable the hair dye composition to provide a visible color change on the darker hair that is substantially the same as the color change on light hair. In some aspects, the use of the primer and hair dye composition in combination may result in a visible color change to dark hair that has ΔE of less than 20 (e.g., 15 or less, 10 or less or even 5 or less) relative to the color change on lighter hair from using the dye alone. Some non-limiting example of primers are AG HAIR COLOUR CARE BB CREAM available from AG, Canada and Pre-Color Primer from L'Oreal, France.

The hair dye compositions herein provide an excellent aesthetic appearance to hair. In particular, the hair dye compositions herein can provide excellent Hue, Saturation, Lightness, and/or Gloss to the hair. “Hue” refers to the color obtained and corresponds to value ranging from 0° to 360° (e.g., 0° (red), 30° (orange), 60° (yellow), 90°, 120° (green), 150°, 180° (cyan), 210°, 240° (blue), 270° (violet), 300° (magenta), or) 330°. “Saturation” is the intensity of the color and has a value ranging from 0% (gray) to 100% (pure color). “Lightness” is the amount of white or black mixed with the color to provide various tints and shades. The Lightness value can be 0% (black) to 100% (white). Gloss is the amount of light reflecting from a surface which gives the effect of shine and can range from 1 to 100, where a lower value indicates lower gloss/higher matte. HSL values can be measured using a spectrophotometer according to the Color Measurement method described in more detail below. Gloss can be measured concurrently with HSL, for example, by setting the SCI/SCE mode on a suitable spectrophotometer to include a specular component (SCI mode) whereby reflected light from a surface is quantified as the Gloss value. Additionally or alternatively, L*a*b* colors values can be measured with a suitable spectrophotometer and converted to HSL color values.

In some aspects, the hair dye compositions herein may be provided to a user in a package that enables the user to activate and/or deactivate the hair dye composition in the package, for example, by irradiating the hair dye composition with EMR through a transparent portion of the package. The hair dye composition, when activated and/or deactivated in its package, may have the same or different color properties from what it has when applied to hair and then activated and/or deactivated. In some instances, the hair dye composition may be exposed to EMR in its package to partially activate and/or deactivate it, and then applied to the hair and exposed to additional EMR to further activate and/or deactivate the hair dye composition.

The hair dye compositions herein enable a user to conveniently change the visible color of their hair to a variety of different colors with a single hair dye composition. The hair dye composition may be tailored to deliver a visible color change based on the fluence of the activating EMR, the wavelength of the activating EMR. the fluence of the deactivating EMR, the wavelength of the deactivating EMR, the activation time of the photochromic dye, and/or the deactivation time of the photochromic dye. In some aspects, the hair dye compositions herein may provide long lasting color change (e.g., more than 2 hours, 4 hours, 8 hours, 12 hours, 18 hours, or even 24 hours or more) upon activation and/or deactivation.

The color of the photochromic dyes in the hair dye composition, when activated, are not particularly limited and may be any color, as desired. In some instances, it may be desirable for the activated dye color to be selected from cyan, magenta, yellow, and combinations thereof to take advantage of CMYK printing principles to provide a wide range of different colors. Cyan may be characterized as having an HSL value of 300°, 100% and 50%. Magenta may be characterized as having an HSL value of 180°, 100% and 50%. Yellow may be characterized as having an HSL value of 60°, 100% and 50%. In some instances, a color that has a Hue value within 3°, a Saturation value within 10%, and a Lightness value within 10% of the target value for a particular color may be considered that color. For example, a color with an HSL value of 178°, 95%, 45% may be considered Cyan.

### Photochromic Dye

Photochromic dyes are generally recognized as dyes that undergo a reversible color change when exposed to EMR of a particular wavelength and fluence (“activating EMR”). Photochromic dyes are classified into two types, p-type and t-type. When a photochromic dye is exposed to an activating and/or deactivating EMR it undergoes a molecular transformation between isomers of the photochromic dye molecule.

The compositions herein include a p-type photochromic dye. The p-type photochromic dyes suitable for use herein can be activated by an activating EMR having a wavelength of 250 nm to 395 nm (e.g., 260 nm to 380 nm or 270 nm to 370 nm) and a fluence of 1 mJ/cm² to 50 J/cm². In some aspects, the hair dye compositions include two or more different photochromic dyes. For example, the hair dye composition may include two or more p-type photochromic dyes, a p-type photochromic dyes and a t-type photochromic dyes, or a combination of photochromic dyes and a non-photochromic hair dye.

P-type photochromic dyes are thermally irreversible, but photochemically reversible. In other words, a p-type photochromic dye will change color when irradiated with activating EMR but will generally not revert back to its original color when the activating EMR is removed or when the temperature of the dye changes. A p-type photochromic dye is deactivated by exposing the dye to deactivating EMR, which has a different wavelength from the activating EMR. In some embodiments, the deactivating EMR has a wavelength of 400 to 700 nm (e.g., 425 nm to 650 nm) and a fluence of 1 mJ/cm² to 50 J/cm². The photochromic dyes suitable for use herein may have an activation and/or deactivation time of less than 1 second up to 10 minutes (e.g., 0.001 seconds to 5 minutes, 0.01 second to 3 minutes, 0.1 second to 2 minutes seconds, 0.5 second to 90 seconds, or even 10 seconds to 60 seconds). Relatively shorter activation and/or deactivation times may be more convenient to a user, and therefore preferred.

In some instances, the p-type photochromic dyes used herein include those belonging to the diarylethene (“DAE”) family and/or fulgide family. Some non-limiting examples of diarylethene and fulgide photochromic dyes are described in U.S. Pat. No. 6,123,952 and US20120017929. Some particularly suitable examples of P-type photochromic dyes for use herein include DAE-0001 (cyan), DAE-0012 (magenta), DAE-0068 (yellow) from Yamada Chemical Company.

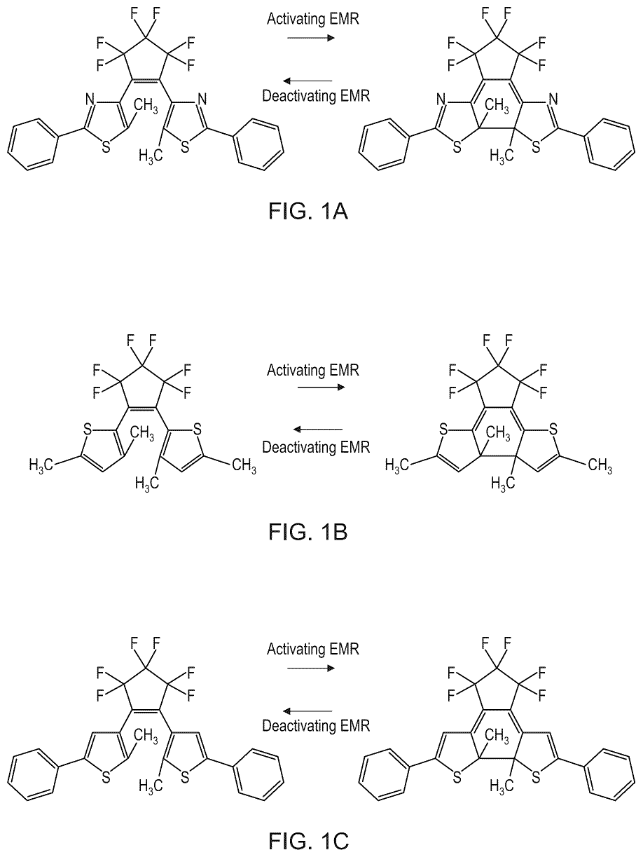

FIGS. 1A, 1B, and 1C depict the chemical structures of diarylethene photochromic dyes. FIG. 1A depicts 4,4′-(perfluorocyclopent-1-ene-1,2-diyl)bis(5-methyl-2-phenylthiazole), which is a p-type photochromic dye that changes from clear to magenta when exposed to a suitable activation energy. FIG. 1B depicts 5,5′-(perfluorocyclopent-1-ene-1,2-diyl)bis(2,4-dimethylthiophene), which is a p-type photochromic dye that changes from clear to yellow when exposed to a suitable activation energy. FIG. 1C depicts 3,3′-(perfluorocyclopent-1-ene-1,2-diyl)bis(2-methyl-5-phenylthiophene), which is a p-type photochromic dye that changes from clear to cyan when exposed to a suitable activating energy. In FIGS. 1A, 1B and 1C, the structures on the left-hand side illustrate the unactivated state of the dye, and the structures on the right-hand side illustrate the activated state of the dye. As illustrated in the figures, the activated dyes have a closed ring structure and the unactivated dye have an open ring structure. The wavelength of the activating EMR for the photochromic dyes illustrated in FIGS. 1A, 1B, and 1C can range from 250 nm to 395 nm, and the wavelength of the deactivating EMR can range from 400 nm to 700 nm, depending on the dye. The fluence of the activating and deactivating EMR of the photochromic dyes can be 1 mJ/cm² to 50 J/cm².

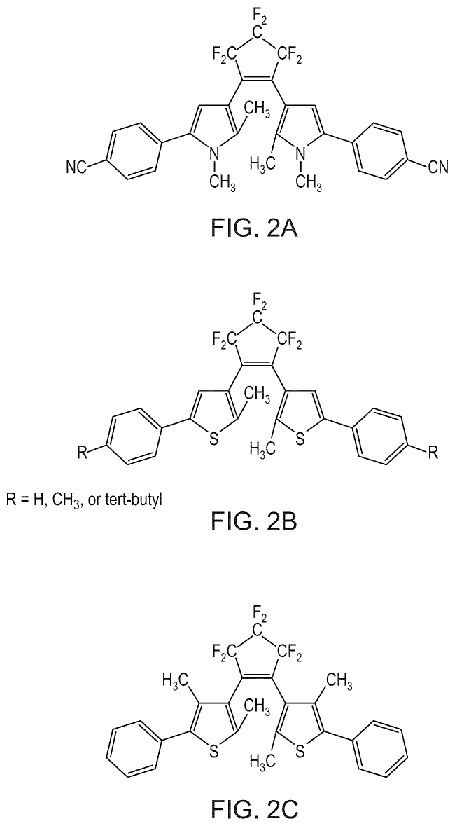

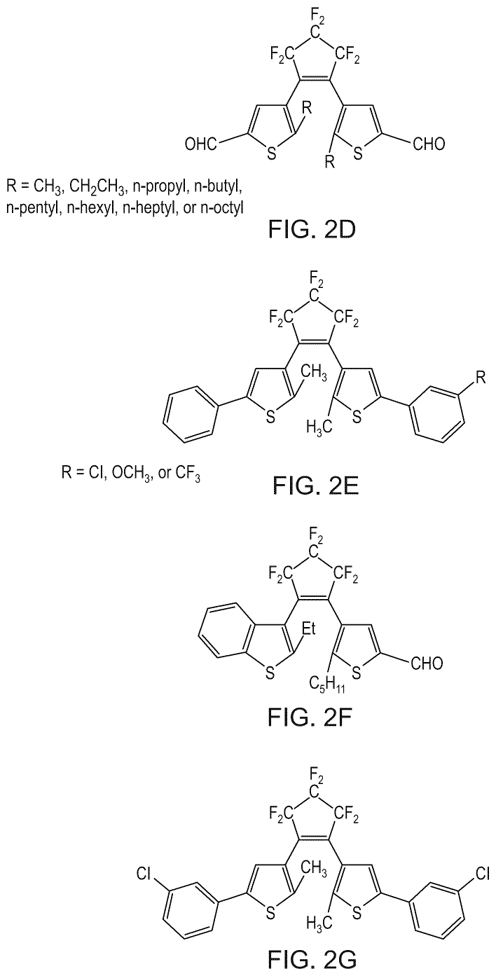

FIGS. 2A to 2G depict additional examples of blue photochromic dyes (i.e., dyes that generally appear blue to the human eye when activated by an activating EMR).

In some embodiments, the hair dye compositions herein may include one or more t-type photochromic dyes. T-type photochromic dyes are thermally reversible, which generally means they will revert back to their original color once the activating EMR is removed. T-type photochromic dyes include azobenzene and spiropyran compounds. Some non-limiting examples of t-type photochromic dyes can be found in U.S. Pat. Nos. 5,581,090, 5,730,961, and FR1604929.

### Carrier

The hair dye composition herein includes 1%-95% of a cosmetically acceptable carrier. The carrier will typically be liquid, but embodiments in which the carrier is solid or semi-solid (e.g., gel) are also contemplated herein. The carrier may include polar and/or non-polar materials and may be in the form of a single phase (e.g., solution or dispersion) or multiple phases (e.g., an oil-in-water or water-in-oil emulsion). In some embodiments, the carrier includes a solvent in which the photochromic hair dye compounds are soluble. The solvent may make up some or all of the carrier (e.g., 5% to 100%). In some embodiments, the photochromic dye has a solubility of 1 mg/L to 200 g/L in the solvent. Some non-limiting examples of solvents that may be suitable for dissolving DAE photochromic dyes include emulsifiers (e.g., Tween® 20, Tween® 40, Tween® 60, and Tween® 80 brand polysorbate and Span® 20, Span® 40, Span® 60 and Span® 80 brand sorbitan laurate), fatty acids (e.g., steric acid, oleic acid, and palmitic acid), fatty alcohols (e.g., cetyl alcohol, lauryl alcohol, stearyl alcohol, oleyl alcohol, and octyldodecanol), fatty esters (e.g., caprylic/capric triglyceride, isopropyl palmitate, and isopropyl myristate), silicones (e.g., cyclopentasiloxane, cyclohexasiloxane, dimethicone, and methicone) and botanical oils (e.g., wheatgerm oil, beauty-leaf oil, sesame oil, macadamia oil, grapeseed oil, rapeseed oil, coconut oil, groundnut oil, palm oil, castor oil, jojoba oil, olive oil or cereal germ oil, cedarleaf oil, turpentine oil, and eucalyptus oil) and combinations of these. Botanical oils may be particularly suitable because they can be naturally and/or sustainably sourced and are generally perceived by consumers as being better for their body.

### Optional Ingredients.

The hair dye compositions herein may include other optional ingredients commonly found in hair dye compositions and/or other cosmetic hair and/or skin care compositions. These additional ingredients may be present at 0.5% to 95%. Some non-limiting examples of optional ingredients can be found in U.S. Pat. No. 7,056,351 and US2002/0053110 and may include non-photochromic hair dyes, stabilizing agents (e.g., UV stabilizing agents), binding agents, deposition aids, organic acids, surfactants, hair or skin conditioning agents, anti-dandruff actives, fungicides, alkyl ethoxylate hydrocarbons, silicone compounds, cationic polymers, proteins, amino acids, preservatives, moisturizing agents, viscosity modifiers, emulsifiers, pH modifiers, buffering agents, chelants, and combinations of these.

### Method of Making

In some embodiments, the hair dye compositions may be made using conventional methods of making such compositions. In some embodiments, it may be desirable to combine the photochromic dyes and a solvent into a premix, which is then added to the carrier and/or other ingredients in the hair dye composition.

### Hair Dye Product

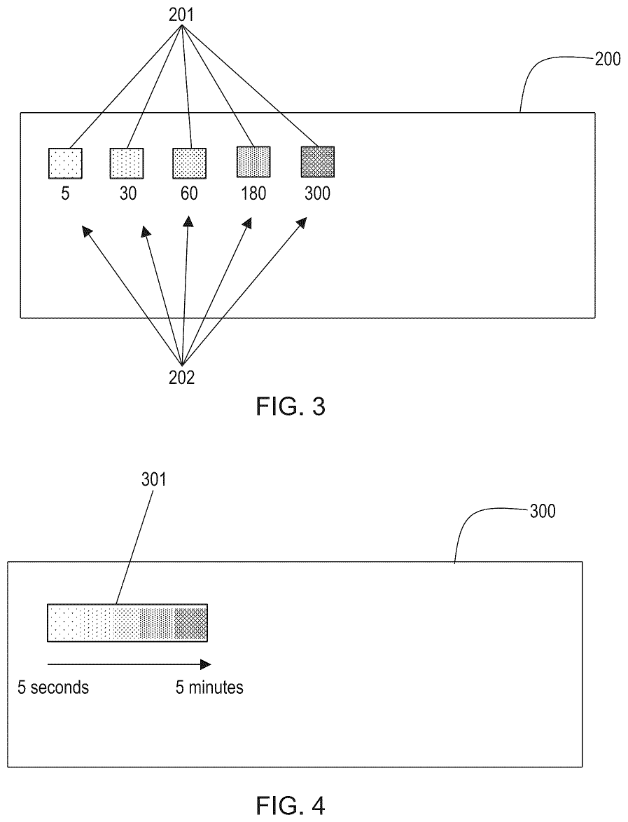

The hair dye compositions herein may be placed in a primary and, optionally, a secondary package and displayed in a retail environment for sale. The material(s) used to make the primary and/or secondary packaging is not particularly limited and can include any suitable material known for use in hair dye packaging. In some aspects, it may be desirable to use sustainably sourced and/or recyclable materials. The package may include indicia that communicate to a consumer that the hair dye product is a tunable hair dye product. For example, the package may depict hair with its natural color and the hair with two or more other colors provided by the hair dye composition. In some aspects, the packaging may include indicia, for example as illustrated in FIG. 3 or FIG. 4, that show a spectrum of hair colors provided the hair dye composition as a function of EMR exposure time and/or fluence.

FIG. 3 illustrates an example of a secondary package 200 in the form of a box. The secondary package 200 is configured to house a primary package containing a hair dye composition. The primary package may be in the form of a bottle, jar, vial or other package suitable for containing a hair dye composition. In some aspects, a primary package and/or hair dye composition may be visible through a window (not shown) in the secondary package 200. In some aspects, it may be desirable to inhibit or prevent EMR from reaching the hair dye composition, for example, by configuring the primary and/or secondary packages to block EMR that can activate and/or deactivate a hair dye composition contained in the primary packaging (e.g., EMR having a wavelength of 200 nm to 700 nm, 250 nm to 700 nm, 400 nm to 700 nm, or 280 nm to 400 nm).

The secondary package 200 may include indicia that communicate the tunable aspect of the hair dye composition. In the example depicted in FIG. 3, the indicia include a series of colored squares 201. Each square 201 is a different color and corresponds to the color of the dye after exposure to activating EMR or deactivating EMR for a particular duration. The indicia in FIG. 3 also include numbers 202 to indicate the duration of EMR exposure that results in the color in each square (i.e., 5 seconds, 10 seconds, 30 seconds, 60 seconds, and 120 seconds).

FIG. 4 illustrates an example of a secondary package 300 in which the tunability of a hair dye composition is depicted as a spectrum 301 of color that can be obtained based on the exposure of the hair dye composition to activating and/or deactivating EMR. In some aspects, the indicia on the package 200, 300 may be positioned near a see-through portion of the package such that a consumer is able to compare a color change of the hair dye composition, for example, due to activation and/or deactivation, to the indicia.

In some aspects, the hair dye product may be provided as a kit that includes two or more photochromic dyes and/or photochromic hair dye compositions that can be combined by a user to form a hair dye mixture that provides a desired hair color. In some aspects, the kit includes two or more hair dye compositions, each comprising a photochromic dye, a suitable solvent, and, optionally, other ingredients commonly included in a hair dye composition. In some aspects, the kit includes two or more photochromic dyes powders that can be combined before or after dissolving the powder in a suitable solvent or carrier. The photochromic hair dye powders and/or hair dye compositions in the kit can be combined at different ratios to provide a variety of different hair colors. The kit may also include a mixing container for combining the photochromic dye powders and/or hair dye compositions. The photochromic dyes may be activated and/or deactivated before and/or after forming a hair dye mixture.

### Array of Hair Dye Products

The hair dye compositions and products described herein can be combined to create an array of tunable hair dye products. The array of hair dye products may be displayed via a merchandise display system in a brick-and-mortar retail store and/or may be available for electronic purchase from a digital retail environment (e.g., online shopping). The array may include two or more distinct but commonly branded (i.e., common brand identifier or common source identifier) tunable hair dye products that are simultaneously offered for sale and presented in a retail setting. The discrete hair dye products may be differentiated by their respective labeling information and/or packaging indicia. They may also respectively include other differentiating product features, such as differences in color, performance, ingredient type and/or ingredient amount.

In some aspects, the array of tunable hair dye products includes a first hair dye product, a second hair dye product and, optionally, one or more additional hair dye products, as desired. Each hair dye product includes a tunable hair dye composition, for example, as described above, disposed in a package. In some aspects, the hair dye compositions provide the same or substantially the same color change to different colors of hair. For example, the array may include a first hair dye composition configured to change light hair (e.g., hair color value of 1 to 3) to red and a second hair dye composition configured to change medium hair (e.g., hair color value of 4 to 6) or dark hair (e.g., hair color value of 7 to 10) to red. In this example, the red color provided by the hair dye products may have an HSL value of 0, 100, 50, and the final red hair color values provided by the first and second hair dye composition in this example should be substantially the same. Substantially the same color change means the ΔE between any of the different hair types (i.e., light, medium and dark) should differ by no more than 20 (e.g., have a ΔE of less than 20, 15, 10, 5, 4, 3, 2, or even less than 1). The dyed hair color value provided by the compositions herein should differ from the original hair color by a ΔE of greater than 1 (e.g., 2 to 100, 5 to 75, 10 to 50, or even 15 to 30). A ΔE of 1 or less is generally not perceptible to the human eye.

One or more of the hair dye products in the array may include a primer or other pre-treatment composition that provides a color improvement benefit to the hair and/or hair dye composition. For example, a hair color primer may be included in the hair dye products of the array intended for use on medium or dark hair. In this example, the primer may include a white pigment that lightens a target portion of hair prior to application of the photochromic dye. In this way, the resulting hair color will be the same or substantially the same for all hair colors (i.e., ΔE of 20 or less). The amount and/or type of primer used may vary for products intended for use on different color hair. For example, products intended for use on light hair may include no primer or a small amount of primer. Products intended for use on medium and dark hair may include incrementally more primer.

In some aspects, the array of hair dye products includes a first hair dye product, a second hair dye product and, optionally, one or more additional hair dye products, configured to provide a different color to the same color of hair. For example, the array may include a first hair dye composition configured to change light hair, medium hair, and/or dark hair to red (e.g., HSL value of 0, 100, 50) and a second hair dye composition configured to change light hair, medium hair, and/or dark hair to green (HSL value of 120, 100, 50). In this example, the final red and green hair color values provided by the first and second hair dye composition should differ from the original hair color and each other by a ΔE of more than 1 (e.g., 2, 3, 4, 5, 10, 20, 30, 50, 75, or more).

In some aspects, the hair dye compositions in the products of the array may include 1 or more of the same or different photochromic dyes at the same or different concentrations and/or ratios. For example, hair dye compositions in different products of the array may include the same photochromic dye at different concentrations. Additionally or alternatively, the hair dye compositions in different products of the array may include two or more of the same photochromic dyes at different ratios to one another. In some aspects, the hair dye compositions in different products of the array may have one or more hair dyes in common and 1 or more that are different.

In some aspects, the hair array may include hair dye products that differ in an active ingredient and/or hair or scalp benefit provided by the hair dye composition. For example, the array may include a hair dye composition formulated to provide an intended tunable color change as well as a hair and/or scalp benefit. Some non-limiting examples of hair and scalp benefits include improved hair strength, decreased hair brittleness, improved hair volume, decreased hair dryness, decreased hair or scalp oiliness, reduced scalp itchiness, reduced scalp irritation/redness, improved hair fragrance, improved hair retention,

The packages of the different hair dye products of the array may include indicia to communicate verbally or non-verbally to a consumer that the product is intended for use on a particular hair color (e.g., light, medium, or dark) to provide the same or substantially the same tunable hair colors. For example, a first product package may include indicia that depict a person with blond hair and also depicts the same person with one or more of the hair colors provided by the hair dye composition in the package. In this example, the array may include a second product package with indicia that is the same as or similar to the indicia of the first product package, except that a person with brown or black hair is depicted. It is to be appreciated that any number of products, product packages and hair dye compositions intended for use on any number of different hair colors are contemplated by the present array.

### Method of Use

The hair dye compositions herein can be applied to a target portion of hair where a color change is desired. The target portion of hair may be wet or dry and may applied by a hair professional or at home. In one example, it may be desirable to wash the hair, but not condition it, and then dry the hair before applying the hair dye composition. In this example, after applying the hair dye composition, the hair dye composition is allowed to remain on the hair, optionally covered (e.g., with a shower cap or the like), for at least 10 minutes (e.g., 15, 20, 25, 30, or even up to 60 minutes) before rinsing the hair with water. The hair may then be irradiated with an activating EMR, such as one of the EMR types described herein, followed by irradiation with a second (or more) activating EMR and/or one or more deactivating EMR, such as one of the deactivating EMR described herein.

In some instances, it may be desirable to activate and/or deactivate the photochromic dye in the hair dye composition prior to applying it to hair. For example, a dye composition comprising two or more photochromic dyes may be placed in a transparent or translucent container and exposed to an activating EMR to change at least one of the dyes to a different color. The activated or partially activated hair dye composition may then be applied to the hair. Additionally or alternatively, the hair dye composition may be exposed to a second activating EMR and/or first deactivating EMR to provide a second color and prior to application to the hair. Further, the activated hair dye composition may be exposed to an additional activating EMR or deactivating EMR after application to hair. This process may be repeated as needed to achieve a desired hair color.

### METHODS

### Color Measurement

This method enables the measurement of both the initial color and color change on hair treated with the hair dye compositions herein. The method uses a suitable spectrophotometer or colorimeter such as an X-Rite Ci7800 sphere benchtop brand spectrophotometer, Konica Minolta CM-3600A brand spectrometer, or equivalent. The value used to express the degree of color change on any particular substrate is Delta E (ΔE), which is calculated using L*, a* and b* values (“Lab values”) according to the following equation:

$$ΔE = √ ( {( {L}_{1} - {L}_{2} )}^{2} + {( {a}_{1} - {a}_{2} )}^{2} + {( {b}_{1} - {b}_{2} )}^{2} ) ,$$

where:
   L₁ is the first L* value and L₂ is the second L* value;
   a₁ is the first a* value and a₂ is the second a* value; and
   b₁ is the first b* value and b₂ is the second b* value.

L* is a measure of lightness and darkness (color intensity), wherein L=100 is white and L=0 is black. The a* value is a measure of the red and green quotients (color hues) in which positive values equate to red and negative values equate to green. The b* value is a measure of the yellow and blue quotients (color hues) in which positive values equate to yellow and negative values equate to blue.

The Lab value measurements are carried out on a full scanning spectrophotometer with a wavelength of from 400-700 nanometers. The spectrophotometer records the color in terms of L* a* and b* values. These values can then be converted into other color systems (such as the HSL color system). The spectrophotometer is set to: mode=SCI/SCE; spot size=10 mm; and light=D65. The sample (e.g., standard hair switch) is placed in a sample holder designed to hold the hair sample in a uniform orientation during measurement and ensure it does not move during measurement. The hair must be spread to cover the 10 mm port during color measurement. Three measurements are run per treatment.

### EXAMPLES

### Example 1: Example Formulations

Table 1 shows hair dye composition formulas that may be suitable for use as hair dye compositions herein.

TABLE 1

| | Product Form | Ingredient list |
|---|---|---|
| Inventive 1 | Oil-based | Isododecane, dimethicone, dimethiconol, diethyl sebacate, isodecyl |
| | | pivalate, tris(ethylhexanoate) glyceryl, coconut oil, DAE dye, |
| | | fragrance, phenoxyethanol, tocopherol, jojoba seed oil, |
| Inventive 2 | Water based | Tween80, coconut oil, water, DAE dye, fragrance |
| Inventive 3 | Mousse | Cyclohexasiloxane, dimethicone, dimethicone/vinyl dimethicone |
| | | crosspolymer, silica, acrylates copolymer, disodium stearoyl |
| | | glutamate, aluminum hydroxide, coconut oil, DAE dye, fragrance |
| Inventive 4 | Solid powder | Talc, dimethicone, HDI/trimethylol hexyllactone crosspolymer, silica, |
| | | dimethicone/vinyl dimethicone crosspolymer, mica, titanium dioxide |
| | | (CI 77891), DAE dye, triethoxycaprylylsilane, ethylhexylglycerin, |
| | | glyceryl caprylate, dimethiconol, iron oxides (CI 77492), aluminum |
| | | hydroxide, alcohol, butylene glycol, iron oxides (CI 77491), |
| | | methicone, tocopherol |
| Inventive 5 | Soft gel | Water, iota carrageenan, tween 80, coconut oil, DAE-dye |
| Primer | Emulsion | Water, isodecyl neopentanoate, dimethicone, dimethicone/bis- |
| | | isobutyl ppg-20 crosspolymer, hydroxyethyl acryalte/sodium |
| | | acryloyldimethyl taurate copolymer, isohexadecane, |
| | | hexamethyldisiloxane, isoceteth-20, polysilicone-11, tocopherol, |
| | | polysorbate 60, trisodium ethylenediamine disuccinate, |
| | | phenoxyethanol, chlorphenesin |

### Example 2: Solubility

Photochromic dyes may exhibit poor solubility in some cosmetic solvents. Thus, it is important to select a solvent that is suitable for use on human skin and hair and enables formulation of a stable hair dye composition with sufficient dye to provide good hair coloring results. Stable composition herein do not exhibit phase separation or formation of precipitate. Table 2 shows the solubility of cyan, magenta and yellow dyes in cosmetically acceptable solvents, i.e., solvent that are generally recognized as safe for use in cosmetics by the U.S. Food and Drug Administration.

TABLE 2

| | Max cyan ¹ | Max magenta ² | Max yellow ³ |
|---|---|---|---|
| | concentration | concentration | concentration |
| | (% w/w) | (% w/w) | (% w/w) |
| Polysorbate 80 | 1.08 | 2.20 | 0.89 |
| Polysorbate 20 | 0.9 | 1.78 | 0.95 |
| Caprylic triglyceride | 0.66 | 0.91 | 3.46 |
| Oleyl alcohol | 1.47 | 0.79 | 1.34 |
| Isopropyl myristate | 4.8 | 2.25 | 3.09 |
| Isopropyl isostearate | 4.95 | 2.32 | 3.41 |

¹ DAE-0001 from Yamada Chemical Company
² DAE-0012 from Yamada Chemical Company
³ DAE-0068 from Yamada Chemical Company

As can be seen in Table 2, fatty esters such as isopropyl myristate and isopropyl isostearate were generally the best solvents overall. However, from these data, caprylic triglyceride appears to be a much better solvent for the yellow dye than for the cyan and magenta dyes, which was unexpected.

### Example 3: Activating EMR and Deactivating EMR for Diarylethene Photochromic Dyes

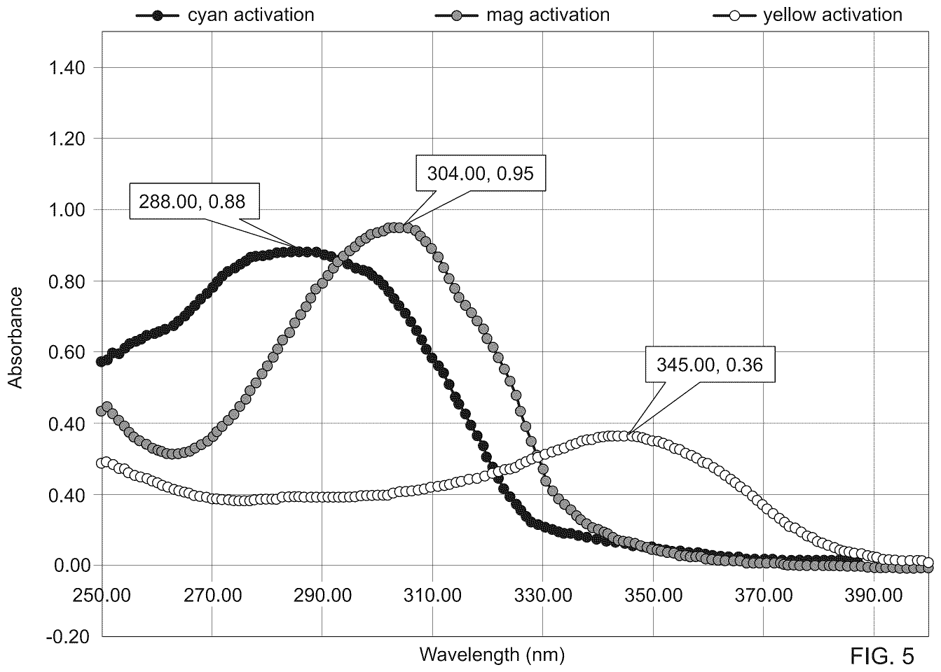

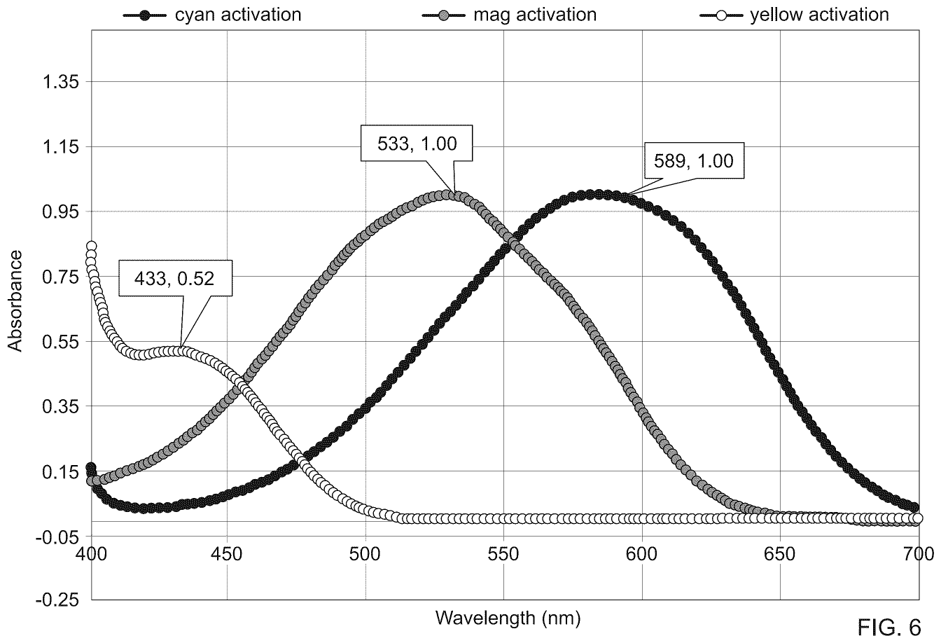

Table 3 shows the activating EMR and deactivating EMR wavelengths for specific p-type photochromic dyes DAE-0001 (cyan), DAE-0012 (magenta), and DAE-0068 (yellow). The activation and deactivation properties are illustrated in FIGS. 5 and 6, respectively. As can be seen in FIG. 5, cyan has a peak activating EMR of between about 270 nm and 300 nm, magenta has a peak activating EMR of between about 290 nm and 310 nm, and yellow has a peak activating EMR of between about 330 nm and 360 nm. As can be seen in FIG. 6, cyan has a peak deactivating EMR of between about 560 nm and 610 nm, magenta has a peak deactivating EMR of between about 510 nm and 540 nm, and yellow has a peak deactivating EMR of between about 410 nm and 450 nm.

TABLE 3

| Hair dye compound¹ | Activating EMR | De-activating EMR |
|---|---|---|
| DAE-0001 (cyan) | 250 nm-320 nm | 500 nm-650 nm |
| DAE-0012 (magenta) | 260 nm-330 nm | 450 nm-600 nm |
| DAE-0068 (yellow) | 310 nm-370 nm | 400 nm-475 nm |

¹From Yamada Chemical Company

### Example 4: Colors Achieved by Different Dye Combinations

This example demonstrates the ability of a p-type photochromic dye to change color. The test compositions are a mixture of DAE photochromic dyes, an isopropyl myristate solvent and a carrier, as shown in Table 4. The dye ratio is based on the weight of cyan dye, unless no cyan dye is present, then it is based on the weight of magenta. In this example, the carrier is Moremo™ Recovery Balm B brand leave-in hair treatment (“Moremo™”), which contains: glycerin, water, dimethicone, butylene glycol, myristyl alcohol, propylene glycol, cyclopentasiloxane, isopropyl myristate, cetyl ethylhexanoate, hydroxypropyl starch phosphate, perfum, steartrimonium chloride, guar hydroxypropyltrimonium chloride, sodium benzoate, cetrimonium chloride, laureth-23, laureth-3, polyquaternium-7, betaine, milk protein extract, panthenol, hydrolyzed collagen, Spirulina platensis extract, Corchorus olitorius leaf extract, disodium EDTA, tocopheryl acetate, C12-15 pareth-3, cocamidopropyl betaine, hydrolyzed keratin, hydrolyzed collagen PG-propyl methylsilanediol, hydrolyzed wheat protein, hydrolyzed corn protein, hydrolyzed soy protein, 1,2-hexanediol, glycine, glutamic acid, lysine, leucine, methionine, valine, serine, cysteine, aspartic acid, alanine, arginine, isoleucine, tyrosine, threonine, phenylalanine, proline, histidine, hydrolyzed silk, propanediol, Ceratonia siliqua (carob) fruit extract, Diospyros kaki fruit extract, Camellia japonica leaf extract, Myrothamnus flabellifolia leaf/stem extract, caprylyl glycol, citric acid, ascorbic acid, oat amino acids, wheat amino acids, soy amino acids, rice amino acids. The test compositions were placed in a 30 mL clear glass vial (available from Thermofisher) and exposed to activating EMR (SUN X11 MAX UV LED brand nail lamp, 365-405 nm and a fluence of 16 J/cm²). HSL values are determined according to the Color Measurement method within 1 minute after removing the activating EMR. The resultant colors are summarized in Table 4. As can be seen in Table 4, the dye combinations provide a wide range of color possibilities.

TABLE 4

| | Carrier | IPM | Total Dye | Dye Ratio (w/w) | | | Resultant Color | | |
|---|---|---|---|---|---|---|---|---|---|
| Ex. | wt % | wt % | wt % | Cyan¹ | Magenta² | Yellow³ | H (°) | S (%) | L (%) |
| 1 | 62.82 | 26.89 | 10.29 | 1 | 1 | 3 | 256 | 25.8 | 43.3 |
| 2 | 61.64 | 26.38 | 11.98 | 1 | 1 | 6 | 230 | 2.59 | 54.5 |
| 3 | 61.60 | 26.37 | 12.03 | 1 | 2 | 3 | 282 | 43.0 | 32.4 |
| 4 | 60.58 | 25.93 | 13.49 | 1 | 2 | 6 | 323 | 10.2 | 49.8 |
| 5 | 65.88 | 28.19 | 5.93 | 0 | 2 | 1 | 343 | 70.6 | 48.0 |
| 6 | 67.17 | 28.75 | 4.08 | 0 | 1 | 1 | 360 | 69.8 | 66.3 |
| 7 | 64.38 | 27.55 | 8.07 | 1 | 0 | 3 | 193 | 20.2 | 47.6 |
| 8 | 62.28 | 26.65 | 11.07 | 2 | 0 | 9 | 213 | 41.3 | 39.4 |
| 9 | 67.15 | 28.74 | 4.11 | 1 | 1 | 0 | 270 | 100 | 10.6 |
| 10 | 65.84 | 28.18 | 5.98 | 1 | 2 | 0 | 283 | 100 | 6.47 |

¹DAE-0001 from Yamada Chemical Company
²DAE-0012 from Yamada Chemical Company
³DAE-0068 from Yamada Chemical Company

### Example 5: Hair Color Change I

This example demonstrates the ability of the inventive hair dye compositions to change the color of hair when an unactivated hair dye composition is applied to hair and then exposed to an activating EMR. In this example, three different hair colors were tested to demonstrate the properties and benefits of the inventive hair dye compositions. The hair colors tested are: medium blonde (hair color value 2), medium light brown (hair color value 5), and black (hair color value 9). Suitable hair switches are available from Kerling International Haarfabrik GmbH, Germany. The hair dye compositions used in this example are composition 5 (C5) from Table 4 and composition A1, which is a 0.5% solution of dye (1:1 weight ratio of cyan and magenta) and acetone.

The initial color of each hair switch is measured according to the Color Measurement method and recorded as the initial color. A first set of test samples were prepared by applying 8 g of composition A1 to the hair tresses in Table 5 (20 g, 8 cm, hair color values 2, 5 and 9) and massaging it through the hair for 1 minute. A second blond test sample was prepared by applying 1 g of dye composition to a blond hair switch (6 g, 8 cm, and hair color value=2) and massaging it through the hair for 1 minute. A second set of brown and black hair test samples were prepared by applying a mixture of 0.25 g dye composition and 0.75 g of L'Oreal® brand hair primer to the surface of the brown and black hair switches (6 g, 8 cm and hair color values 5 and 9). 30 minutes after applying the dye composition to the hair switches, the test samples were exposed to activating EMR (SUN X11 MAX UV LED brand nail lamp, 365-405 nm and a fluence of 16 J/cm²). The final hair color of the test samples was measured according to the Color Measurement method. For the C5 samples, gloss was not measured, but ΔE relative to the blond hair sample is determined. The results are summarized in Table 5.

TABLE 5

| | | Initial color | | | | Final Color | | | | |
|---|---|---|---|---|---|---|---|---|---|---|
| Hair Color/Value | Dye | H (°) | S (%) | L (%) | Gloss | H (°) | S (%) | L (%) | Gloss | ΔE |
| Medium blonde/2 | A1 | 38.9 | 19 | 60 | 24 | 249 | 7 | 41 | 16 | 29.4 |
| Medium light brown/5 | A1 | 20 | 27 | 35 | 7 | 21 | 20 | 20 | 7 | 14.6 |
| Black/9 | A1 | 245 | 9 | 29 | 4 | 226 | 15 | 28 | 5 | 3.2 |
| Medium blonde/2 | C5 | 38.9 | 19 | 60 | — | 349 | 19.8 | 43.5 | — | 31.0 |
| Medium light brown/5 | C5 | 20 | 27 | 35 | — | 348 | 13.7 | 30 | — | 15.8 |
| Black/9 | C5 | 245 | 9 | 29 | — | 322 | 11.4 | 32.7 | — | 14.3 |

### Example 6: Hair Color Change II

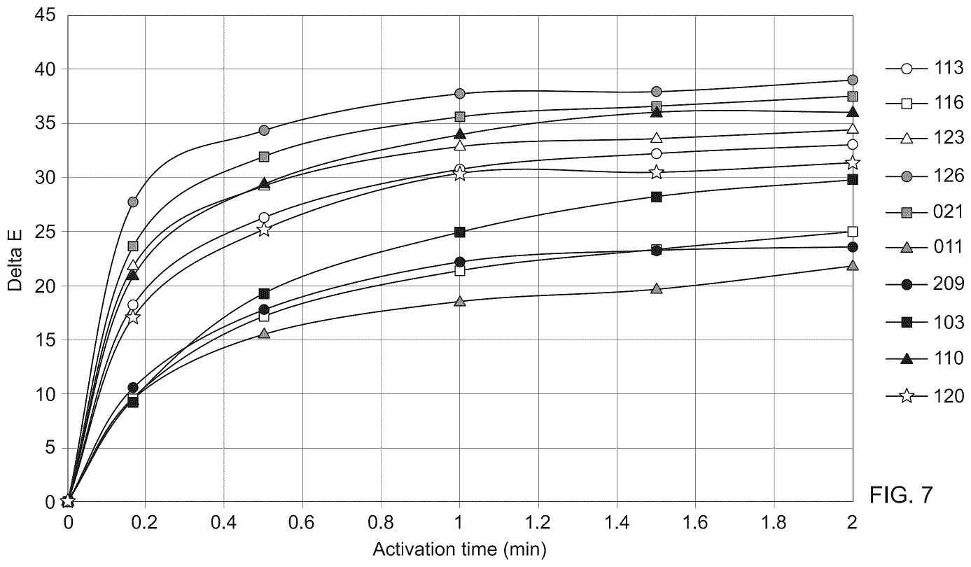

The example demonstrates the ability of the present hair dye compositions to tunably change the color of hair when an unactivated hair dye composition is applied to blond hair (hair color value 2) and then exposed to an activating EMR (365-405 nm) for 2 minutes. Hair switch samples are prepared as described above in Example 5. The test compositions are solutions of 4%-14% total dye, 25%-28% isopropyl myristate solvent and 60%-67% Moremo™ carrier. The hair color change is measured according to the Color Measurement method. The color difference between the initial hair color (i.e., undyed hair color) and the final hair color is shown as AE. The results are summarized in Table 6 below and illustrated in FIG. 7. The numbers in the legend of FIG. 7 correspond to the weight ratio of the dyes shown in Table 6.

TABLE 6

| | | | Resultant Color | | | |
|---|---|---|---|---|---|---|
| Mass ratio of dye | | | (2 min. of activation) | | | |
| Cyan ¹ | Magenta ² | Yellow ³ | H (°) | S (%) | L (%) | ΔE |
| 1 | 1 | 3 | 332 | 7.42 | 44.9 | 33.0 |
| 1 | 1 | 6 | 12.9 | 5.60 | 51.0 | 25.1 |
| 1 | 2 | 3 | 335 | 7.69 | 43.3 | 34.5 |
| 1 | 2 | 6 | 350 | 7.50 | 47.1 | 31.5 |
| 0 | 2 | 1 | 349 | 19.8 | 43.5 | 37.6 |
| 0 | 1 | 1 | 13.2 | 21.2 | 53.7 | 21.9 |
| 2 | 0 | 9 | 32.7 | 4.49 | 52.0 | 23.6 |
| 1 | 0 | 3 | 30.0 | 4.91 | 52.2 | 24.3 |
| 1 | 1 | 0 | 304 | 6.91 | 42.5 | 36.1 |
| 1 | 2 | 0 | 313 | 9.00 | 39.2 | 39.1 |

¹ DAE-0001 from Yamada Chemical Company
² DAE-0012 from Yamada Chemical Company
³ DAE-0068 from Yamada Chemical Company

As can be seen in Table 6, the inventive compositions can provide a wide range of clearly noticeable color changes to hair as demonstrated by a ΔE of greater 20 for all the test samples. Further, as seen in FIG. 7, all the dyes achieve a suitable ΔE in less than 2 minutes (e.g., less than 90 seconds, less than 60 seconds, less than 45 seconds, and even less than 30 seconds), which is desired by consumers.

### Example 7: Hair Color Change III

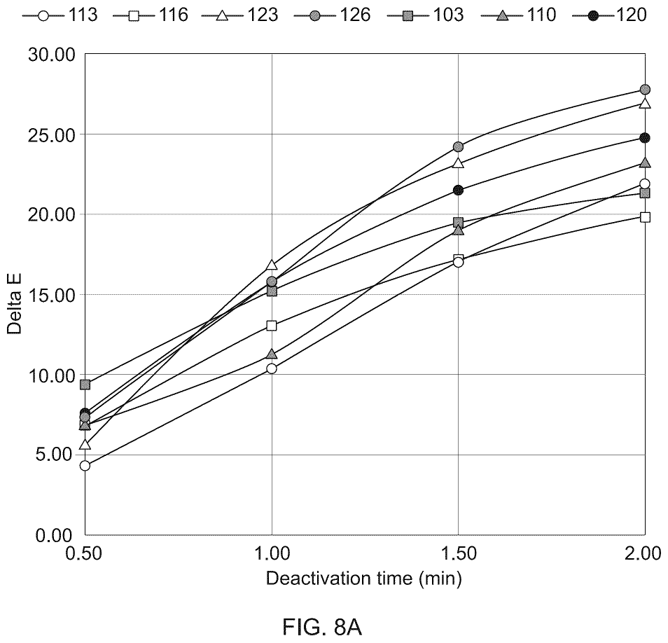

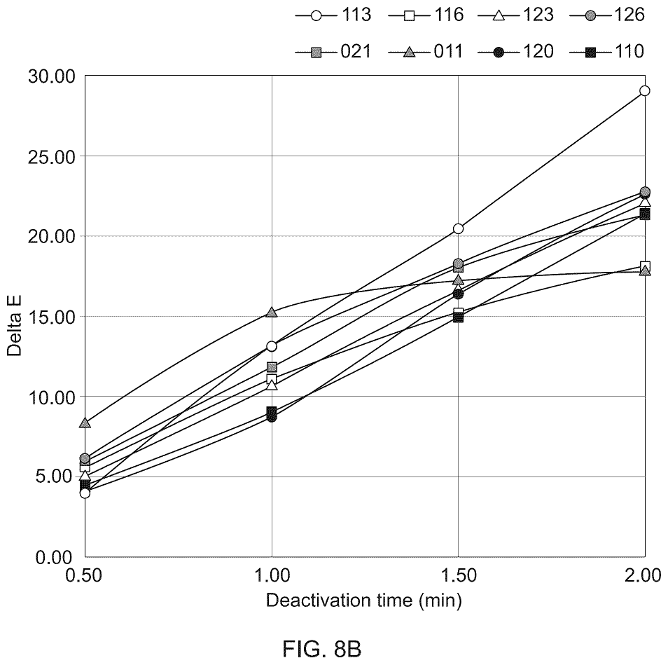

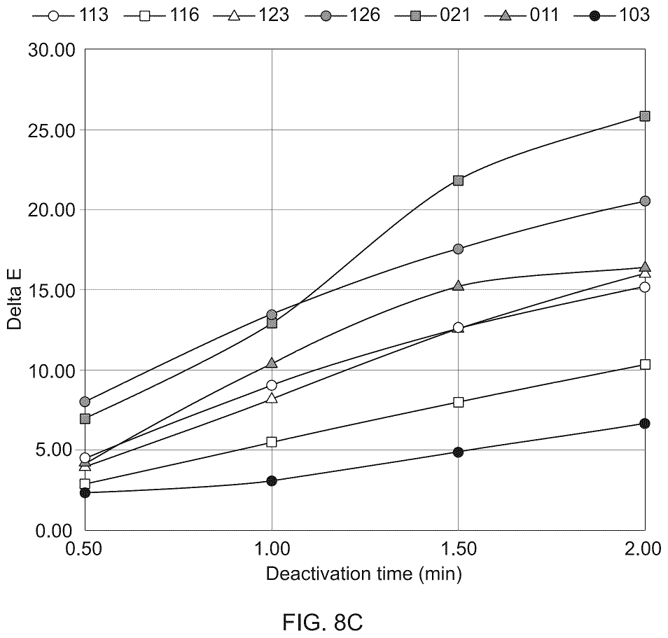

The example demonstrates the ability of the present hair dye compositions to tunably change the color of hair when an activated hair dye composition is exposed to a deactivating EMR. Hair switch samples were prepared as described above in Example 6 using the dyes shown in Tables 7A, 7B and 7C. The test compositions are solutions of 0.5% total dye in an isopropyl myristate solvent. The activated color change was measured according to the Color Measurement method and recorded. The dyed (i.e., activated) hair samples were exposed to a deactivating EMR for 1 minute and the resulting color measured according to the Color Measurement method and recorded. ΔE was calculated as described above. The colors in this example are characterized by their Lab values. The results are summarized in Table 7A, 7B and 7C. FIGS. 8A, 8B and 8C illustrate the deactivation times for the samples based on ΔE. The numbers in the legend in Tables 8A, 8B and 8C correspond to the weight ratio of the dyes shown in Tables 7A, 7B and 7C, respectively.

TABLE 7A
Cyan (Deactivating EMR λ = 589.5 nm, 100% saturation)

| | | | Initial Hair Color (after | | | Final Hair Color (after | | | |
|---|---|---|---|---|---|---|---|---|---|
| Mass ratio of dye | | | 2 min. of activation) | | | 1 min. of deactivation) | | | |
| Cyan | Magenta | Yellow | L | a | b | L | a | b | ΔE |
| 1 | 1 | 3 | 50.5 | 7.5 | 0.7 | 62.2 | 3.3 | 10.4 | 15.8 |
| 1 | 1 | 6 | 56.3 | 3.5 | 6.0 | 65.6 | 1.6 | 15.0 | 13.0 |
| 1 | 2 | 3 | 49.7 | 6.8 | 0.9 | 62.3 | 3.6 | 11.4 | 16.7 |
| 1 | 2 | 6 | 47.5 | 6.9 | −0.8 | 60.3 | 3.8 | 8.0 | 15.9 |
| 0 | 2 | 1 | — | — | — | — | — | — | — |
| 0 | 1 | 1 | — | — | — | — | — | — | — |
| 1 | 0 | 3 | 54.1 | 0.0 | 4.1 | 66.5 | 0.2 | 13.0 | 15.2 |
| 1 | 1 | 0 | 49.0 | 7.5 | −1.9 | 57.4 | 4.5 | 5.0 | 11.3 |
| 1 | 2 | 0 | 44.4 | 9.6 | −3.1 | 53.2 | 7.5 | 2.0 | 10.4 |

TABLE 7B
Magenta (Deactivating EMR λ = 533 nm, 100% saturation)

| | | | Initial Hair Color (after | | | Final Hair Color (after | | | |
|---|---|---|---|---|---|---|---|---|---|
| Mass ratio of dye | | | 2 min. of activation) | | | 1 min. of deactivation) | | | |
| Cyan | Magenta | Yellow | L | a | b | L | a | b | ΔE |
| 1 | 1 | 3 | 50.3 | 7.0 | 1.2 | 59.5 | 3.1 | 7.5 | 11.8 |
| 1 | 1 | 6 | 56.8 | 3.0 | 6.4 | 64.9 | 1.2 | 13.8 | 11.1 |
| 1 | 2 | 3 | 49.6 | 6.9 | 0.9 | 58.2 | 3.8 | 6.5 | 10.7 |
| 1 | 2 | 6 | 49.7 | 7.8 | 1.3 | 60.0 | 3.6 | 8.2 | 13.1 |
| 0 | 2 | 1 | 46.6 | 16.7 | 5.0 | 57.3 | 12.9 | 11.7 | 13.2 |
| 0 | 1 | 1 | 58.3 | 10.8 | 13.5 | 68.8 | 4.0 | 22.1 | 15.3 |
| 1 | 0 | 3 | — | — | — | — | — | — | — |
| 1 | 1 | 0 | 49.0 | 7.4 | −1.6 | 55.8 | 4.1 | 3.4 | 9.0 |
| 1 | 2 | 0 | 45.5 | 8.9 | −2.0 | 52.9 | 6.0 | 1.6 | 8.7 |

TABLE 7C
Yellow (Deactivating EMR λ = 433 nm, 100% saturation)

| | | | Initial Hair Color (after | | | Final Hair Color (after | | | |
|---|---|---|---|---|---|---|---|---|---|
| Mass ratio of dye | | | 2 min. of activation) | | | 1 min. of deactivation) | | | |
| Cyan | Magenta | Yellow | L | a | b | L | a | b | ΔE |
| 1 | 1 | 3 | 49.7 | 7.7 | 0.2 | 57.2 | 3.2 | 2.2 | 9.0 |
| 1 | 1 | 6 | 56.0 | 2.8 | 5.1 | 60.9 | 1.4 | 7.0 | 5.5 |
| 1 | 2 | 3 | 49.2 | 7.1 | 0.6 | 56.2 | 3.4 | 2.2 | 8.1 |
| 1 | 2 | 6 | 48.2 | 8.2 | 0.2 | 59.3 | 2.0 | 4.8 | 13.5 |
| 0 | 2 | 1 | 45.1 | 17.9 | 3.9 | 56.3 | 14.1 | 9.0 | 12.9 |
| 0 | 1 | 1 | 59.7 | 11.9 | 12.2 | 66.1 | 6.0 | 17.9 | 10.4 |
| 1 | 0 | 3 | 56.6 | 0.1 | 4.7 | 59.5 | 0.9 | 4.9 | 3.0 |
| 1 | 1 | 0 | — | — | — | — | — | — | — |
| 1 | 2 | 0 | — | — | — | — | — | — | — |

As can be seen in Tables 7A-7C and FIGS. 8A-8C, the inventive compositions herein exhibit a noticeable color change in less than 1 minute when exposed to a suitable deactivating EMR.

### Example 8: Hair Color Change IV

This example demonstrates the ability of the hair dye compositions herein to tunably change the color of hair when applied to hair and then exposed to an activating EMR, followed by exposure to a deactivating EMR. Two compositions were tested in this example, TC1 and TC2. TC1 is a 0.5% solution of dye (1:1 w/w ratio of cyan and magenta) in acetone. TC2 is a dye solution in an isopropyl myristate solvent and Moremo™ carrier.

A first hair sample was prepared by applying 8 g of TC1 to a medium blond hair tress (20 g, 8 cm and hair value=2). A second hair sample was prepared by apply 1 g of TC2 to a medium blond hair tress (6 g, 8 cm, hair value=2). After 30 minutes, the hair samples were exposed to activating EMR. In this example, the top portion of the activated medium-blonde test hair switch was exposed to deactivating EMR having a wavelength of 500 nm to 600 nm and a fluence of 1.2 J/cm², while the bottom portion of the hair switch was shielded from the deactivating EMR. The bottom portion of the activated test hair switch was then exposed to a deactivating EMR of 600 nm to 700 nm and a fluence of 1.2 J/cm², while the top portion was shielded from the applied deactivating EMR. The HSL values of the top and bottom portions of the hair switch were determined according to the Color Measurement method and the ΔE was calculated. For the hair sample treated with TC1, gloss was also measured. The results are summarized in Table 8 below. The ΔE after activation was determined relative to the original hair color, and the ΔE after deactivation was determined relative to the activated hair color.

TABLE 8

| | After activation | | After deactivation | | After deactivation | |
|---|---|---|---|---|---|---|
| Color | at 250-350 nm | | at 500-600 nm | | at 600-700 nm | |
| Property | TC1 | TC2 | TC1 | TC2 | TC1 | TC2 |
| H (°) | 262.6 | 8.2 | 218.8 | 348.1 | 268.5 | 346.5 |
| S(%) | 10.2 | 97.4 | 1.4 | 95.0 | 6 | 93.98 |
| L(%) | 24 | 10.0 | 32.4 | 13.3 | 31.6 | 13.81 |
| Gloss | 10.8 | — | 10.8 | — | 16.3 | — |
| ΔE | 53.0 | 80 | 13.5 | 9.8 | 9.3 | 21.6 |

As can be seen in Table 8, activating the hair dye composition provides a first color change, and the deactivating steps provide second and third color changes.

### Example 9: Hair Color Change V

This example provides another demonstration of the ability of the hair dye compositions herein to tunably change the color of hair when the dye composition is exposed to an activating EMR followed by exposure to one or more deactivating EMR. In this example, a test hair switch was prepared as described above and dyed with Composition A1 from Example 5. The dyed hair switch was exposed to an activating EMR having a wavelength of 250-350 nm (UV light) and a fluence of 16 J/cm², followed by exposure to a first deactivating EMR of 600-700 nm (red light) and then a second deactivating EMR of 500-600 nm (green light). The results of the testing are summarized in Table 9 below.

TABLE 9

| | | | Hair | | |
|---|---|---|---|---|---|
| | | | color | | |
| | | Hair color | after | Hair color | Hair Color |
| | | after | activation | after | after |
| | Original | application | at | deactivation | deactivation |
| Color | hair | of | 250-350 | at | at |
| Property | color | hair dye | nm | 600-700 nm | 500-600 nm |
| H (°) | 36 | 38 | 236 | 346 | 240 |
| S(%) | 40 | 40 | 11 | 12 | 1 |
| L(%) | 76 | 73 | 51 | 58 | 68 |

As can be seen in Table 9, activation and deactivation of the dye produced noticeably different hair colors.

### Example 10: Hair Color Change VI

This example provides another demonstration of the ability of the hair dye compositions herein to tunably change the color of hair when the dye composition is exposed to an activating EMR prior to applying the dye to the hair. Composition A1 from Example 5 (cyan+magenta) was also used in this test. However, the photochromic dyes were activated with an activating EMR (250-350 nm, 12 J/cm²) prior to applying it to the hair. After the activated dye composition was applied to the hair, it was exposed to a deactivating EMR (600-700 nm, 1.2 J/cm²). The results of the testing are summarized in Table 10 below.

TABLE 10

| | | Original | Hair dye | Hair color | |
|---|---|---|---|---|---|
| | Original | hair dye | composition | after | Hair color |
| Color | hair | composition | color after | application | after |
| Property | color | color | activation | of dye | deactivation |
| H (°) | 38 | 46 | 234 | 20 | 25 |
| S(%) | 51 | 34 | 23 | 18 | 27 |
| L(%) | 71 | 80 | 33 | 33 | 42 |

As can be seen in Table 10, activating the dye prior to application to the hair yields a different color versus applying the dye and then activating. Deactivating the dye also produces a different color change relative to dye that is activated after application to the hair. In this example, the hair dye composition may be completely deactivated (i.e., returned to its original clear and substantially colorless form) and the activated again to produce the color change effect demonstrated in Example 4 or 5. Thus, a single hair color composition herein could provide 6 or more different hair colors.

### Example 11: Hair Color Change VII

This example demonstration of the ability of the hair dye compositions herein to provide the same color change to hair of different colors. In this example, light blond hair (hair color value 1) and brown hair (hair color value 6) were treated with a hair dye composition intended to change the color of the hair to blue/cyan. After application to the hair, the dye was exposed to activating EMR (250-350 nm, 16 J/cm²). The resulting hair color was measured according to the Color Measurement method. The results of the testing are summarized in Table 11.

TABLE 11

| | Hair dye | | | | |
|---|---|---|---|---|---|
| Hair color (value) | composition used | H (°) | S(%) | L(%) | ΔE |
| Light Blond (1) | Inventive 3 | 243 | 16 | 78 | 2.56 |
| Medium Brown (6) | Primer + Inventive 2 | 240 | 20 | 78 | |

As can be seen in Table 11, the hair dye compositions provided substantially the same color of hair when used on blond and brown hair.

### Example 12: Composition Formulation

The example demonstrates the importance of balancing the amount of solvent and dye to achieve suitable color changing properties without sacrificing in-use, hair-feel properties. Test compositions comprising varying amounts of cyan dye (DAE-0001 from Yamada Chemical Company) and isopropyl myristate or magenta dye (DAE-0012 from Yamada Chemical Company) and isopropyl myristate are shown in Tables 12A and 12B, respectively. The balance of the composition is a Moremo™ carrier. One gram of the test composition is applied to a blond (hair value=2) hair switch as described above and exposed to an activating EMR for two minutes. The dyed hair samples were then evaluated for acceptable or unacceptable color accuracy (i.e., how close is the resulting color to the desired color), dye distribution (i.e., how evenly is the resulting color distributed throughout the sample) and oiliness. Desirable results are indicated as “good.” Undesirable results are indicated as “bad.”

TABLE 12A
Cyan

| | Wt % | | | | | | | |
|---|---|---|---|---|---|---|---|---|
| Dye | 0.2 | 0.4 | 0.61 | 0.7 | 0.81 | 1.0 | 1.2 | 1.47 |
| Isopropyl | 9.8 | 19.6 | 30.1 | 34.3 | 39.8 | 49.0 | 58.8 | 29.2 |
| myristate | | | | | | | | |

Results

| | | | | | | | | |
|---|---|---|---|---|---|---|---|---|
| Color | bad | bad | bad | good | good | good | good | good |
| Accuracy | | | | | | | | |
| Dye | bad | bad | good | good | good | good | good | good |
| distribution | | | | | | | | |
| Oiliness | good | good | good | bad | bad | bad | bad | good |

As can be seen in Table 12A, it may be desirable to formulate the hair dye compositions to include more than 0.4 wt % cyan dye (e.g., 0.5% to 10%, 06% to 5%, 0.7% to 3%, 0.8% to 2% or even 0.9% to 1.5%) and less than 35% (e.g., 20% to 33%, 25% to 32%, 27% to 31%, or even about 29% to 30%) isopropyl myristate or other fatty ester solvent.

TABLE 12B
Magenta

| | Wt % | | | | | | | |
|---|---|---|---|---|---|---|---|---|
| Dye | 0.2 | 0.4 | 0.61 | 0.7 | 0.81 | 1.0 | 1.2 | 1.4 |
| Isopropyl | 9.8 | 19.6 | 30.1 | 34.3 | 39.8 | 49.0 | 58.8 | 68.6 |
| myristate | | | | | | | | |

Results

| | | | | | | | | |
|---|---|---|---|---|---|---|---|---|
| Color | bad | bad | bad | good | good | good | good | good |
| Accuracy | | | | | | | | |
| Dye | bad | bad | good | good | good | good | good | good |
| distribution | | | | | | | | |
| Oiliness | good | good | good | good | bad | bad | bad | bad |

As can be seen in Table 12B, it may be desirable to formulate the hair dye compositions to include more than 0.6 wt % magenta dye (e.g., 0.62% to 10%, 065% to 5%, 0.7% to 3%, 0.8% to 2% or even 0.9% to 1.5%) and less than 40% (e.g., 20% to 35%, 25% to 34%, 27% to 33%, or even about 28% to 32%) isopropyl myristate or other fatty ester solvent.

It is believed, without being limited by theory, that tailoring the individual concentration of each dye to be between 0.7% and 2% provides particularly suitable color vibrancy, activation time and hair feel.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An array of tunable hair dye products for providing the same hair color to users with different hair colors, comprising:
a first hair dye product comprising a package and a dye composition contained in the package, wherein the hair dye composition comprises a p-type photochromic dye and a carrier; and
a second hair dye product comprising a package and a hair dye composition contained in the package, wherein the hair dye composition comprises a p-type photochromic dye and a carrier,
wherein the first and second hair dye compositions, when activated, yield different hair color changes to hair of the same color and the same color change to hair of different colors, according to the Color Measurement method.

2. The array of claim 1, wherein the first and second products are spatially separated from each other in a retail environment.

3. The array of claim 1, wherein the first and second products are sold individually.

4. The array of claim 1, wherein the first hair dye product package and the second hair dye product package display a common brand.

5. The array of claim 1, wherein a concentration of the photochromic dye in the first hair dye product is different from a concentration of the photochromic dye in the second hair dye product.

6. The array of claim 1, wherein the photochromic dye in the first hair dye product and the photochromic dye in the second hair dye product comprise different photochromic dyes.

7. The array of the claim 6, further comprising a third hair dye product comprising a package and a dye composition contained in the package, wherein the hair dye composition comprises a p-type photochromic dye and a carrier, and the photochromic dye in the third hair dye product is different from the photochromic dye in the first hair dye product and the photochromic dye in the second hair dye product.

8. The array of claim 1, wherein a ΔE between the hair color change provided by the first hair dye composition to hair having a first color and the hair color change provided by the second hair dye composition to hair having a second color is less than 15, according to the Color Measurement method.

9. The array of claim 8, wherein the ΔE is less than 10.

10. The array of claim 1, wherein the photochromic dyes are activatable by an activating EMR having a wavelength of about 250 nm to about 395 nm.

11. The array of claim 1, wherein the photochromic dyes are deactivatable by a deactivating EMR having a wavelength of about 400 nm to about 700 nm.

12. The array of claim 1, wherein the first and second hair dye compositions each comprise a fatty ester solvent.

13. The array of claim 12, wherein the solvent is isopropyl myristate, isopropyl isostearate or a mixture thereof.

14. An array of tunable hair dye products formulated to provide the same tunable hair color to users who have different hair colors, comprising:
a) a first tunable hair dye product comprising a first hair dye composition contained in a first package, wherein the first hair dye composition comprises first and second p-type photochromic dyes present at a weight ratio of first photochromic dye to second photochromic dye of 1:10 to 10:1;
b) a second tunable hair dye product comprising a second hair dye composition contained in a second package, wherein the second hair dye composition comprises the first and second photochromic dyes present at a weight ratio that is different from the weight ratio of the first hair dye composition;
c) wherein the first package and the second package are spatially separated from each other in a retail environment such that the first package and the second package are visible to a consumer during the consumer's purchasing decision process.

15. The array of claim 14, wherein a ΔE between the hair color change provided by the first tunable hair dye product, when activated, and the hair color change provided by the second tunable hair dye product, when activated, is less than 20, according to the Color Measurement method.

16. The array of claim 14, wherein the first product and the second product display a common brand.

17. The array of claim 14, wherein the first and second photochromic dyes are activatable by an activating EMR have a wavelength of about 250 nm to about 395 nm.

18. The array of claim 14, wherein the first and second photochromic dyes are deactivatable by a deactivating EMR have a wavelength of about 400 nm to about 700 nm, and wherein the deactivating EMR of the first photochromic dye is different from the deactivating EMR of the second photochromic dye.

19. The composition of claim 14, further comprising a third package comprising a third hair dye composition, wherein the third hair dye composition comprises the first and second photochromic dyes present at a weight ratio that is different from the weight ratios of the photochromic dyes in the first and second hair dye compositions.

20. An array of tunable hair dye products formulated to provide the same hair color to users with different hair colors, comprising:
a) a first package comprising a first hair dye composition, wherein the first hair dye composition comprises at least one photochromic dye that changes the hair dye composition to a first color when activated by an activating EMR; and
b) a second package comprising a second hair dye composition, wherein the second hair dye composition comprises the at least one photochromic dye and changes the second hair dye composition to a second color when activated by an activating EMR, wherein the first and second colors have a ΔE of more than 1 according to the Color Measurement method.
